(19) United States
(12) Patent Application Publication
(10) Pub. No.: US 2022/0027726 A1
(43) Pub. Date: Jan. 27, 2022

(54) PRE-PROCESSING DEVICE, PRE-PROCESSING METHOD, AND PRE-PROCESSING PROGRAM

(71) Applicant: Nippon Telegraph and Telephone Corporation, Tokyo (JP)

(72) Inventors: Jumpei Yamashita, Tokyo (JP); Hidetaka Koya, Tokyo (JP); Hajime Nakajima, Tokyo (JP)

(21) Appl. No.: 17/296,692

(22) PCT Filed: Nov. 15, 2019
(86) PCT No.: PCT/JP2019/044884

§ 371 Date: May 25, 2021

(51) Int. Cl. G06N 3/08

(52) U.S. Cl. CPC G06N 3/08; G06F 17/18

(57) ABSTRACT

A preprocessing unit (130) of a training device (10) include: a pre-training data collection unit (131) configured to collect pre-training data including continuous input data measured in an environment simulating an estimation environment and output data corresponding to the continuous input data; and a conversion unit (132) configured to convert the continuous input data into continuous input data pieces of a plurality of sizes including a size larger than the input data, convert the output data corresponding to the continuous input data into output data pieces respectively corresponding to the continuous input data pieces of the plurality of sizes, and output the continuous input data pieces of the plurality of sizes and the output data pieces as training data.

### TECHNICAL FIELD

The present invention relates to a preprocessing apparatus, a preprocessing method, and a preprocessing program.

### BACKGROUND ART

A machine learning technique has been proposed enabling the output value to be estimated robustly even if the input value is highly non-linear data or data with a large amount of noise. For example, a neural network (NN) or a convolutional neural network (CNN) is used to solve the problem of estimating one output value corresponding to sequential inputs at a certain interval.

To solve the problem of estimating one output value from sequential values in a certain interval with the CNN, the CNN first needs to learn an association between “input sequence within interval” and an “output value” successfully measured in the past. Then, once the learning is completed, the learned model can estimate an unknown “output value” in response to a new “input sequence in interval” input thereto. The size of the interval is an important factor in this context. The CNN may be provided with data pieces corresponding to intervals of various different lengths as inputs.

For example, a variation in size of the input sequence can be absorbed by preprocessing including: preparing an input unit equivalent to the maximum input sequence; and when input data smaller than that is to be input, padding the periphery of the data with 0. When the number of units is fixed, there is an option other than 0 padding. Specifically, a target range may be designated using attention mechanism. With the mechanism, the input unit also includes values in the periphery of the input sequence, and the values are classified into positive and negative values. Furthermore, the number of units may be variable, and a difference in the number of units may be absorbed by a special pooling layer.

### CITATION LIST

### Non Patent Literature

Non Patent Literature 1: Hideki Nakayama “Extraction of image features and transfer learning using deep layer convolutional neural network” The Institute of Electronics, Information and Communication Engineers Speech Committee, July Conference, 2015

### SUMMARY OF THE INVENTION

### Technical Problem

When the size (the length in a case of one dimensional array for example) of the interval of an input sequence at the time of learning is assumed to be A, the CNN is trained to be capable of accurately estimating the output for “input sequence with size A”. Thus, there is a problem in that the CNN provided with an input sequence with a size B different from that at the time of learning is provided for estimation, cannot perform the estimation appropriately. FIG. 15 is a diagram illustrating sizes of intervals of input sequences at the time of learning and at the time of estimation. As illustrated in FIG. 15, in a case where the length of the input sequence at the time of learning is 6, when input sequences with a length 6 and a length 4 different from the length at the time of learning are input to the CNN at the time of estimation, the estimation cannot be appropriately performed for the length 4 which is different from that at the time of learning, and an output is diverged (see (1) and (2) in FIG. 15).

In order to avoid this, a sequence of the same size as the sequence used at the time of estimation needs to be used as training data FIG. 16 is a diagram illustrating sizes of intervals of input sequences at the time of learning and at the time of estimation. If the lengths of the input sequences at the time of learning include 4 and 6, the estimation can be appropriately performed for the input sequences with the lengths 4 and 6 at the time of estimation (see (1) and (2) in FIG. 16).

Unfortunately, there are many cases where a sequence of the same size as that used at the time of estimation is unable to be collected as training data. The input sequence is data continuous in time or space. Such continuous data may a result of sectionalizing larger continuous data at an equal interval. Generally, to acquire values with fine granularity sectionalized temporary/spatially for measuring a certain item, a more sophisticated measurement device or method is required. Generally, such a sophisticated measurement device or method is expensive.

Thus, an output value finely sectionalized at a level that is originally desired as output data might not be acquired. FIG. 17 is a diagram illustrating input data and output data to and from the CNN. FIG. 17 illustrates an example in which the input data is one-dimensional sequence data such as time-series data. An original intention may be to sectionalize input continuous data with fine granularity as in data Da, so that the CNN can learn outputs for the respective short input sequences (see (1) in FIG. 17). However, an output value finely sectionalized at a level originally intended as the output may not be acquired. Furthermore, due to technical or economic conditions required to be satisfied for the measurement, only an output corresponding to a largely sectionalized input sequence might be a practical option. In such a case, as in data Db, only the large input sequence and an output corresponding thereto can be learned (see (2) in FIG. 17).

As described above, the related-art method has been plagued by a problem in that input sequences of fine granularity and corresponding outputs may not be available for learning. The related-art method has been further plagued by a problem in that an estimation may fail to be appropriately performed when an input sequence of a size different from that at the time of learning is input at the time of estimation.

Transfer learning is a solution for a problem (a major example of which including overfitting) due to a failure to collect a sufficient amount of data in an actual environment for use. This solution features the use of a model that has been trained using data acquired in an environment simulating the actual environment. Unfortunately, there is no related-art transfer learning that can be applied to solve the problem in a situation completely lacking the data on the size/length of a target in the actual environment.

This because the target of the transfer learning, which can compensate for data lacking in the actual environment, is the input sequences of the same size. In other words, the transfer learning is implemented with the number of input units of the network being the same between the pre-training and weak retraining in the actual environment, and requires the size (interval) of the images input thereto to be the same.

As an example, an example case of an actual environment will be described in which only a limited amount of input data of length 2 and corresponding output data are collectable. In this case, in order to solve the problem, a large amount of input data with a length 2 and corresponding output data are collected and pre-trained in a simulated environment. Then, weak retraining is performed using the limited amount of input data with the length 2 acquired in the actual environment and corresponding output data, to improve the estimation accuracy. Thus, with the existing transfer learning, the estimation cannot be appropriately performed if output data corresponding to the input data with the length 2 cannot be obtained at all at the time of retraining in the actual environment.

The present invention is made in view of the above, and an object of the present invention is to provide a preprocessing apparatus, a preprocessing method, and a preprocessing program enabling acquisition of training data with which pre-training of a model can be appropriately performed, even when the size is different between the input data at the time of estimation in the actual environment and the input data for pre-training.

### Means for Solving the Problem

A preprocessing apparatus according to the present invention to solve the problem described above and achieve the object include: a collection unit configured to collect pre-training data comprising continuous input data measured in an environment simulating an estimation environment and output data corresponding to the continuous input data; and a conversion unit configured to convert the continuous input data into continuous input data pieces of a plurality of sizes including a size larger than the input data, convert the output data corresponding to the continuous input data into output data pieces respectively corresponding to the continuous input data pieces of the plurality of sizes, and output the continuous input data pieces of the plurality of sizes and the output data pieces as training data.

### Effects of the Invention

With the present invention, training data with which a pre-training of a model can be appropriately performed can be acquired, even when the size is different between the input data at the time of estimation in the actual environment and the input data for pre-training.

### BRIEF DESCRIPTION OF DRAWINGS

FIG. 1 is a diagram illustrating an example of a configuration of an estimation system according to a first embodiment.

FIG. 2 is a diagram illustrating input/output data to/from a CNN model.

FIG. 3 is a diagram illustrating a related-art learning method.

FIG. 4 is a diagram illustrating processing in a training device.

FIG. 5 is a diagram illustrating processing in an estimation apparatus.

FIG. 6 is a flowchart illustrating a procedure of pre-training processing executed by the training device.

FIG. 7 is a flowchart illustrating a procedure of retraining processing executed by the estimation apparatus.

FIG. 8 is a diagram illustrating an eyeball movement estimation method based on related-art Electrooculography (EOG).

FIG. 9 is a diagram illustrating pre-training for an eyeball movement estimation method using the EOG according to Example 1.

FIG. 10 is a diagram illustrating retraining for an eyeball movement estimation method using the EOG according to Example 1.

FIG. 11 is a diagram illustrating images captured by a camera.

FIG. 12 is a diagram for explaining a sight position estimation method using an image captured by a camera in the related art.

FIG. 13 is a diagram for explaining pre-training in the sight position estimation using an image captured by a camera according to Example 2.

FIG. 14 is a diagram illustrating an example of a computer that realizes the training device and the estimation apparatus by executing a program.

FIG. 15 is a diagram illustrating sizes of intervals of input sequences at the time of learning and at the time of estimation.

FIG. 16 is a diagram illustrating sizes of intervals of input sequences at the time of learning and at the time of estimation.

FIG. 17 is a diagram illustrating input data and output data to and from the CNN.

### DESCRIPTION OF EMBODIMENTS

Hereinafter, embodiments of the present invention will be described in detail with reference to the drawings. The present invention is not limited to the embodiment. Further, in description of the drawings, the same parts are denoted with the same reference signs.

### Embodiment 1

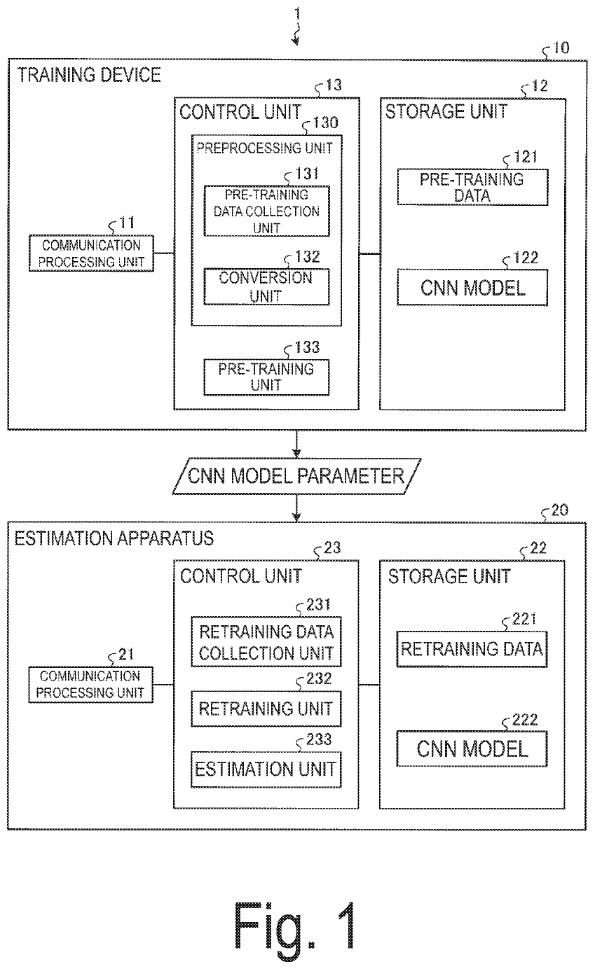

First, a first embodiment of the present invention will be described. FIG. 1 is a diagram illustrating an example of a configuration of an estimation system according to the first embodiment. As illustrated in FIG. 1, an estimation system 1 according to an embodiment includes a training device 10 and an estimation apparatus 20.

The training device 10 pre-trains a model used by the estimation apparatus 20. The training device 10 pre-trains the model using, as pre-training data, continuous sequential input data measured in an environment simulating an estimation environment and output data corresponding to the continuous sequential input data. The input data in the pre-training data is data with a finer granularity than the input data input to the estimation apparatus 20 in the actual environment, that is, data having a size smaller than that of the input data input to the estimation apparatus 20. The training device 10 outputs the model parameters of the pre-trained model to the estimation apparatus 20.

The estimation apparatus 20 is an apparatus provided in the actual environment, and estimates one output value corresponding to the continuous sequential input data as the estimation target, using the pre-trained model in the training device 10. Furthermore, before the estimation, the estimation apparatus 20 performs transfer learning (retraining) which is weak training using retraining data collected in the actual environment. The retraining data comprises continuous sequential input data collected in the actual environment and output data corresponding to the input data, and is data with a coarser granularity, that is, a larger size than the input data collected as the pre-training data by the training device 10.

Configuration of Training Device

Next, a configuration of the training device 10 will be described. The training device 10 includes a communication processing unit 11, a storage unit 12, and a control unit 13.

The communication processing unit 11 is a communication interface that transmits and receives various types of information to and from another apparatus (the estimation apparatus 20 for example) connected via a network or the like. The communication processing unit 11 is implemented by a network interface card (NIC) or the like, and performs communication between another apparatus and the control unit 13 (which will be described below) via an electrical communication line such as a local area network (LAN) or the Internet.

The storage unit 12 is realized by a semiconductor memory element such as a random access memory (RAM) or a flash memory or a storage apparatus such as a hard disk or an optical disk, and a processing program for causing the training device 10 to operate, data used during execution of the processing program, and the like are stored in the storage apparatus.

The storage unit 12 has pre-training data 121 and a CNN model 122.

The pre-training data 121 comprises continuous sequential input data and output data corresponding to the continuous sequential input data, measured in an environment simulating an estimation environment. The input data of the pre-training data 121 is data measured in an environment simulating the estimation environment, and is data with a finer granularity than the input data input to the estimation apparatus 20 in the actual environment. The pre-training data 121 includes the size of the retraining input data in at least one estimation environment, as the size of the continuous input data. The pre-training data 121 has a dataset including an indicator enabling a pre-training algorithm to determine the data of the size equal to that of the retraining input data in the estimation environment, so that an operation can be performed with the retraining input data having an impact that is equal to or larger than that of data with other sizes, on a pre-training process.

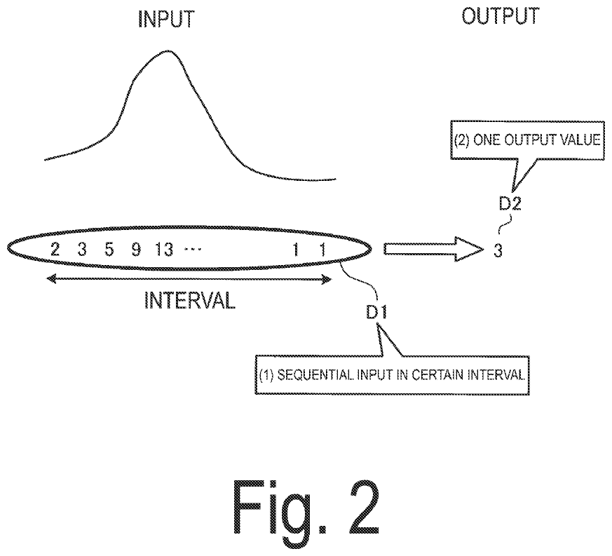

The CNN model 122 is a model adopting CNN. FIG. 2 is a diagram illustrating input/output data to and from the CNN model 122. As illustrated in FIG. 2, upon receiving serial input data D1 in a certain interval, the CNN model 122 solves the problem of estimating one output value, and outputs an output value D2 (see (1) and (2) in FIG. 2). The CNN model 122 estimates an output corresponding to unknown input data by learning the input/output relationship of the data. The CNN model 122 includes various parameters of the model that has learned continuous sequential input data and output data.

Note that the model used in the present embodiment is not limited to the CNN model. The model used in the present embodiment can be any model that can be trained to be capable of estimating the output data from continuous sequential input data.

The control unit 13 controls the entire training device 10. The control unit 13 includes an internal memory for storing a program that defines various processing procedures or the like as well as required data, and executes various processes using the programs and the data. For example, the control unit 13 is an electronic circuit such as a central processing unit (CPU) or a micro processing unit (MPU). Further, the control unit 13 functions as various processing units by operating various programs. The control unit 13 includes a preprocessing unit 130 and a pre-training unit 133.

The preprocessing unit 130 executes preprocessing described below on the pre-training data 121 of the CNN model 122, to provide training data with which the CNN model can be appropriately pre-trained, even when the size differs between the input data at the time of estimation in the actual environment and the input data of the pre-training data. The preprocessing unit 130 includes a pre-training data collection unit 131 (collection unit) and a conversion unit 132.

The pre-training data collection unit 131 collects pre-training data comprising the continuous input data measured in an environment simulating the estimation environment and output data corresponding to the continuous input data. The pre-training data collection unit 131 collects pre-training data including, as the size of the continuous input data, the size of the retraining input data in at least one estimation environment, and having a dataset including an indicator enabling the pre-training algorithm to determine the data with the size equal to that of the retraining input data in the estimation environment.

The conversion unit 132 converts the continuous input data collected by the pre-training data collection unit 131 into continuous input data pieces of a plurality of sizes including a size larger than that of the input data. The conversion unit 132 converts output data corresponding to the continuous input data collected by the pre-training data collection unit 131 into output data pieces respectively corresponding to the continuous input data pieces of a plurality of sizes. The conversion unit 132 outputs the pre-training data comprising the input data and the output data as a result of the conversion, to the pre-training unit 133.

The conversion unit 132 converts the continuous input data, in accordance with a distribution in which the number of the retraining input data pieces in the estimation environment collected by the pre-training data collection unit 131 is equal to or larger than the number of other input data pieces with a size different from that of the retraining input data. The distribution corresponds to a probability distribution in which the number of retraining input data pieces is larger than the number of input data pieces of other sizes. This probability distribution has a convex shape having the size of the input data used in the estimation environment at the center of the distribution, for the sake of increasing the estimation accuracy as much as possible with the data size in the estimation environment.

The pre-training unit 133 trains the CNN model 122 with continuous input data pieces of a plurality of sizes and output data pieces respectively corresponding to the continuous input data pieces of a plurality of sizes, as a result of the conversion by the preprocessing unit 130. The pre-training unit 133 outputs various parameters of the CNN model 122 that has learned the large amount of pre-training data as a result of the conversion by the preprocessing unit 130 to the estimation apparatus 20 in the actual environment.

Configuration of Estimation Apparatus

Next, a configuration of the estimation apparatus 20 will be described. The estimation apparatus 20 is an apparatus provided in an actual environment, and includes a communication processing unit 21, a storage unit 22, and a control unit 23.

The communication processing unit 21 has a function that is similar to that of the communication processing unit 11, and is a communication interface that transmits and receives various types of information to and from another apparatus (the training device 10 for example) connected via a network or the like.

The storage unit 22 has a function similar to that of the storage unit 12, is implemented by a semiconductor memory device such as a RAM or a flash memory or a storage device such as a hard disk or an optical disk, and stores a processing program for operating the estimation apparatus 20, data used during execution of the processing program, and the like. The storage unit 22 has retraining data 221 and a CNN model 222.

The retraining data 221 comprises continuous input data and output data corresponding to the input data that are collected for the retraining in the actual environment. The retraining input data is data with a coarser granularity than the input data as the pre-training data for the training device 10, that is, data with a larger size than the input data input to the training device 10.

The CNN model 222 is configured with various parameters output from the training device 10 as model parameters, and then is retrained by weak training in the estimation apparatus 20.

The control unit 23 controls the entire estimation apparatus 20. The control unit 23 has a function similar to that of the control unit 13, and is an electronic circuit such as a CPU or an MPU. The control unit 23 includes a retraining data collection unit 231, a retraining unit 232, and an estimation unit 233.

The retraining data collection unit 231 collects the retraining data comprising continuous sequential input data and output data corresponding to the input data, both of which are collected in the actual environment. These retraining data pieces are data with a larger size than the input data collected as pre-training data in the training device 10.

The retraining unit 232 additionally trains the CNN model 222 with the weak training using the retraining data to update the model parameters of the CNN model 222. For example, the retraining unit 232 implements the weak training with a learning coefficient set to be low at portions of the CNN model 222 that are distant from the output layer. In the estimation system 1, pre-training of the CNN model is performed in advance using a large amount of data acquired in the environment simulating the actual environment, and then the CNN model is additionally trained by weak training using a small amount of data acquired in the actual environment. This allows the estimation system 1 to generate the CNN model 222 that can perform estimation with high accuracy while avoiding overfitting, even when only a small amount of data can be obtained in the actual environment.

The estimation unit 233 performs estimation using the retrained CNN model 222. The estimation unit 233 uses the CNN model 222 to estimate one output value corresponding to the continuous sequential input data as the estimation target.

Processing Flow

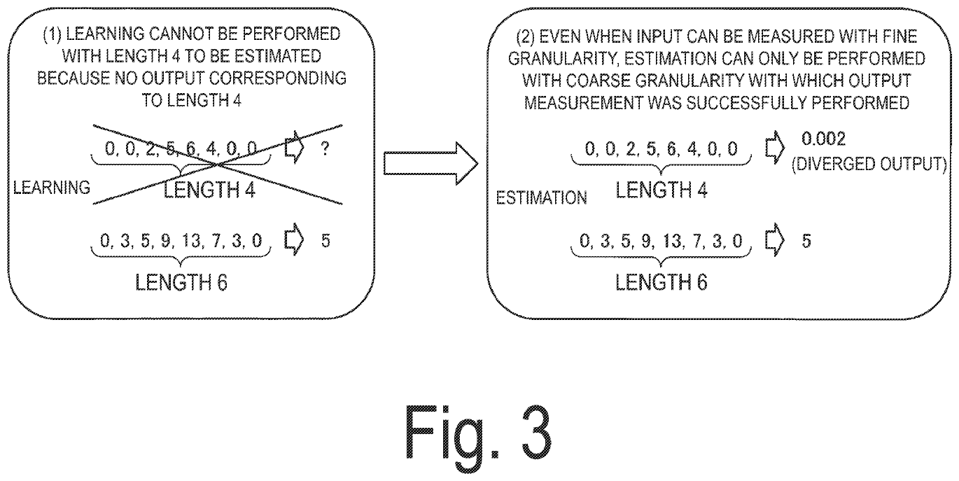

Now, a related-art learning method will be described. FIG. 3 is a diagram illustrating the related-art learning method. In the related art, there has been a problem in that the estimation cannot be appropriately performed if the input data input to the CNN model at the time of estimation has a size different from that at the time of the pre-training. Specifically, when an output corresponding to the length (e.g., 4) desired to be estimated fails to be obtained for the pre-training, training cannot be performed with data of length 4 (see (1) in FIG. 3). In this case, even if the input data can be measured with a fine granularity (for example, length 4), the estimation can only be performed using input data having a coarser granularity (for example, length 6) with which the output has been successfully measured (see FIG. 3 (2)). As a result, even when the input data of the length 4 is input to the CNN model at the time of estimation, the output is diverged, and thus the estimation cannot be appropriately performed. As described above, in the related art, the estimation has been unable to be performed appropriately, when the input sequence of a size different from that at the time of training is input at the time of estimation.

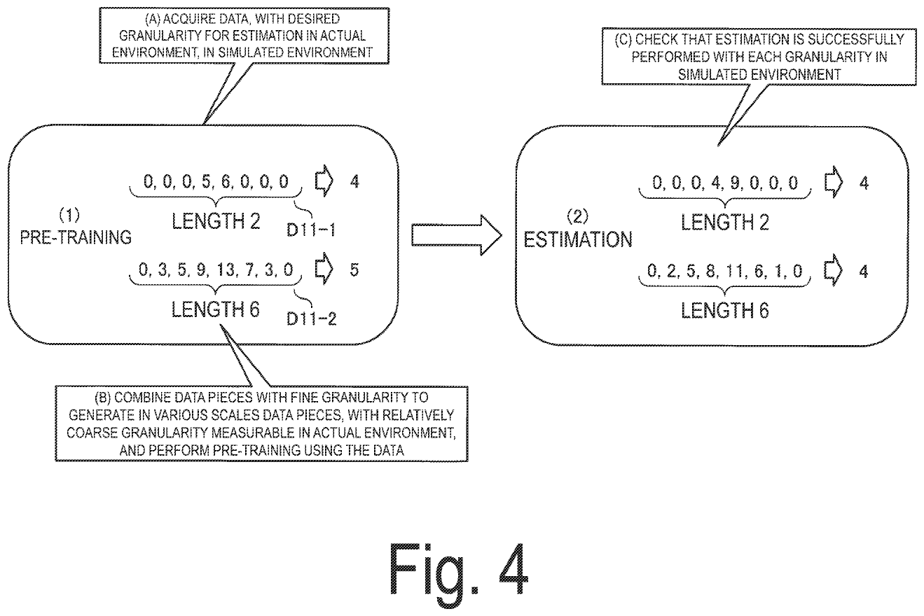

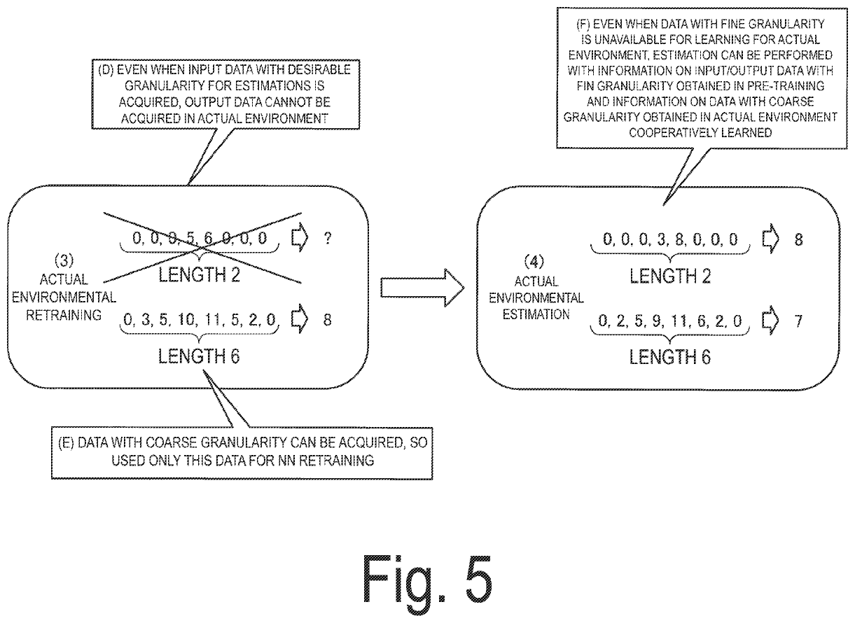

On the other hand, in the training device 10 according to the present embodiment, the preprocessing unit 130 converts the pre-training data so that model can perform the pre-training appropriately, even when the size differs between the input data at the time of estimation in the actual environment and the input data in the pre-training data. FIG. 4 is a diagram illustrating processing in the training device 10. FIG. 5 is a diagram illustrating processing in the estimation apparatus 20.

As illustrated in (1) in FIG. 4, the training device 10 acquires, under a simulated environment, data with a desired granularity for estimation in the actual environment (see (A) in FIG. 4). In this process, for example, the training device 10 collects pre-training data D11-1 comprising input data of length 2 and corresponding output data “4”, by performing measurement with a fine granularity. Here, the desired granularity of the input data to be retrained and estimated in the actual environment is assumed to be of length 6. Thus, the collected input data and the input data estimated in the actual environment are different from each other in length.

In this case, in the training device 10, the preprocessing unit 130 combines data pieces with a fine granularity to generate, on various scales, data with a relatively coarse granularity measurable in the actual environment, and uses this data for the pre-training (see (B) in FIG. 4). For example, the preprocessing unit 130 converts the pre-training data 121 into the input data of the length 6 to be estimated in the actual environment and the output data corresponding to the length 6. Data D11-2 thus obtained by the conversion is used in the pre-training.

Then, the pre-training unit 133 trains the CNN model 222 using the pre-training data D11-1 as well as the data D11-2 obtained by the conversion by the preprocessing unit 130, and confirms that the estimation with each granularity is successfully performed in the simulated environment as illustrated in (2) in FIG. 4 (see (C) in FIG. 4).

As described above, the training device performs the pre-training using input data pieces of the length 4 and the length 6, obtained by converting the input data of the length 2 measured in the simulated environment, for example, and performs the retraining with the length 6 in the actual environment. In this process, with the training device, if the number of input data pieces of the length 2 in the pre-training is less than the number of input data pieces of the length 4 and the number of input data pieces of the length 6 in the pre-training, the algorithm erroneously determines that the estimation is successful as long as the estimation is successfully performed appropriately for the input data pieces of the lengths 4 and 6 (network reducing error functions). This is because when the training device performs no operation of making the impact on the training device different among data sizes, the number of data pieces of the length 2 is not regarded as being so significant in the training. Thus, in the pre-training, the number of input data pieces of the length 2 has to be equal to or larger than those of the input data pieces of the lengths 4 and 6.

With the number of input data pieces of the length 2 set to be large, the model can be generated with the input data of the length 2 used in the actual environment regarded as being significant. Still, this does not mean that a larger number of input data pieces of the length 2 always lead to a better result. For example, the training and estimation should not be successful when the number of input data piece of each of the lengths 4 and 6 is one whereas the number of input data pieces of the length 2 is 100. The reason is as follows. Specifically, the model presumes a condition where the input data pieces of the lengths 2, 4, and 6 are input with a certain level of evenness. Thus, the weak training performed in the actual environment using the input data of the length 6 has completely no impact on a route of estimation performed with the input data of the length 2 input to the model. In other words, the model will fail to perform meaningful training or estimation for the input data of the lengths 4 and 6.

Thus, the number of data sizes needs to conform to a uniform distribution (the number of data pieces is the same among the lengths 2, 4, and 6). Even when the objective to conform with the data lengths in the actual environment is to be pursued, the number of data sizes in the actual environment needs to be set to obtain a probability distribution with a convex shape (a distribution such as the number of input data pieces with the length 2 is the largest, and the number decreases as it gets far from this length (in this example, the lengths 4 and 6)).

Even when the number of data pieces fails to be set in this manner, the training device can perform an operation, in the pre-training, of imposing heavier penalty regarding an error for the length of the input data matching the length of the input data in the actual environment, than that imposed on the lengths of other input data pieces. Thus, an operation can be performed in the pre-training so that the input data with the length corresponding to that in the actual environment can be regarded as if it is equally significant as the input data pieces of other lengths. To implement such a method, the conversion unit 132 converts the pre-training data, with the dataset including information (indicator) with which “the input data of a length same as that in the actual environment” can be determined. Note that when preprocessing of padding the periphery with 0 is employed, the part excluding 0 corresponds to the length of the input data, and thus the input data itself serves as the indicator for the determination.

Then, the estimation apparatus 20 receives the model parameters of the CNN model after the pre-training, and performs retraining in the actual environment (see (3) FIG. 5). In the actual environment, even when the input data of a desired fine granularity (the length 2 for example) for estimation is acquired, the output data corresponding to this input data with the fine granularity cannot be acquired (see (D) in FIG. 5). Still, the input and output data of a coarse granularity can be obtained in the actual environment. Thus, the estimation apparatus 20 uses this data to retrain the CNN (see (E) in FIG. 5). For example, the estimation apparatus 20 performs weak training (retraining) using the input data of the length 6 and output data “8” corresponding to the input data.

Thus, as a result of pre-training by the training device 10, as illustrated in (4) in FIG. 5, the estimation apparatus 20 can appropriately estimate the output data corresponding to the length 2 input data. Furthermore, as a result of the pre-training and the weak training based on the retraining data by the training device 10, the estimation apparatus 20 can estimate output data corresponding to the actual environment, also for the input data of the length 6.

Thus, according to the present embodiment, even in a case where fine granularity data cannot be used in retraining in the actual environment, the estimation can be performed for input data of any of the sizes, with information on the input/output data with a fine granularity obtained in the pre-training and information on the data with a coarse granularity obtained in the actual environment learned in a cooperative manner (see (F) in FIG. 5).

Pre-Training Processing Procedure

Next, the pre-training processing procedure will be described. FIG. 6 is a flowchart illustrating a procedure of pre-training processing executed by the training device 10.

As illustrated in FIG. 6, in the training device 10, the pre-training data collection unit 131 of the preprocessing unit 130 collects the pre-training data comprising the continuous sequential input data obtained by the measurement with a fine granularity in the simulated environment and output data corresponding to the continuous sequential input data (step S1). The pre-training data collection unit 131 collects data with a granularity finer than that of the input data input to the estimation apparatus 20 in the actual environment.

Next, in the preprocessing unit 130, the conversion unit 132 executes conversion processing of converting the continuous input data collected in step S1 into continuous input data pieces of a plurality of sizes including a size larger than that of the input data, and converting the output data corresponding to the continuous input data into output data pieces respectively corresponding to the continuous input data pieces of the plurality of sizes (step S2). The conversion unit 132 outputs the pre-training data comprising the input data and the output data as a result of the conversion, to the pre-training unit 133. In this process, the conversion unit 132 converts the continuous input data, in accordance with a distribution in which the number of the retraining input data pieces in the estimation environment collected by the pre-training data collection unit 131 is equal to or larger than the number of other input data pieces with a size different from that of the retraining input data. Furthermore, the conversion unit 132 makes the indicator enabling the determination as described above included, so that the input data with the length that is the same as that in the actual environment can have a large impact in the pre-training.

The pre-training unit 133 performs the pre-training to train the CNN model 122 using the data collected by the pre-training data collection unit 131, as well as continuous input data pieces of a plurality of sized and output data pieces respectively corresponding to the continuous input data pieces of a plurality of sizes, which have been obtained by the conversion by the preprocessing unit 130 (step S3). The pre-training unit 133 outputs various parameters of the CNN model 122 that has learned the large amount of pre-training data including the data as a result of the conversion by the preprocessing unit 130 to the estimation apparatus 20 in the actual environment (step S4).

Retraining Processing Procedure

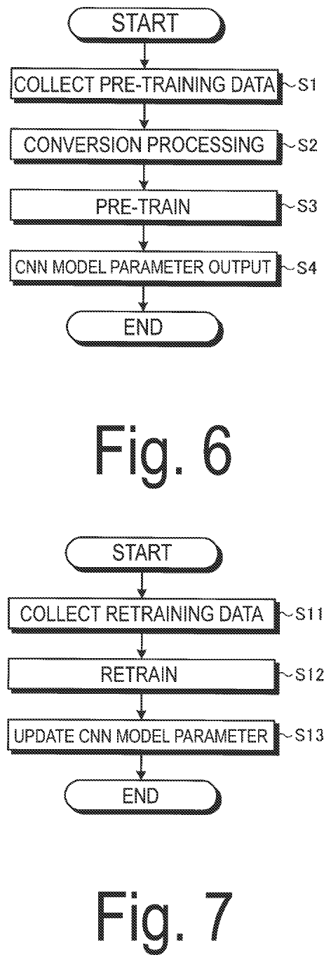

Next, a retraining processing procedure will be described. FIG. 7 is a flowchart illustrating a procedure of retraining processing executed by the estimation apparatus 20.

As illustrated in FIG. 7, in the estimation apparatus 20, the retraining data collection unit 231 collects the retraining data comprising continuous sequential input data and output data corresponding to the input data that are collected under the actual environment (step S11). The retraining data is data with a larger size than the input data collected as pre-training data in the training device 10.

The retraining unit 232 performs retraining with the retraining data to additionally train the CNN model 222 by weak training (step S12) Then, the retraining unit 232 updates the model parameters of the CNN model 222 (step S13). The estimation unit 233 performs an estimation for the input data using the CNN model 222 after retraining.

### Effect of Embodiment

As described above, in the embodiment, the training device 10 that pre-trains the CNN model 122 is provided with the preprocessing unit 130, so that the data collected for the pre-training is pre-processed, and then the pre-training is performed.

Specifically, the preprocessing unit 130 collects pre-training data comprising the continuous input data measured in an environment simulating the estimation environment and output data corresponding to the continuous input data. The preprocessing unit 130 executes preprocessing of converting the continuous input data into continuous input data pieces of a plurality of sizes including a size larger than that of the input data, and converting the output data corresponding to the continuous input data into output data pieces respectively corresponding to the continuous input data pieces of the plurality of sizes. These data pieces are output as training data. The preprocessing unit 130 converts the continuous input data collected by the pre-training data collection unit 131 to a size of at least input data for retraining by the estimation apparatus 20 in the estimation environment.

In other words, the training device 10 executes preprocessing including: combining input data pieces of the pre-training data; converting the input data of the pre-training data into data pieces of a plurality of sizes including a size of the input data at the time of the estimation in the actual environment; and converting the collected output data into output data pieces respectively corresponding to the continuous input data pieces of a plurality of sizes.

Specifically, in the embodiment, the training device 10 is configured to perform the pre-training with the preprocessing unit 130 executing, at the time of pre-training, the processing to generate input data pieces of a plurality of sizes including a size of the input data at the time of the estimation in the actual environment and output data pieces respectively corresponding to the input data pieces, when the output data corresponding to the size of the input data as the pre-training data cannot be obtained at the time of retraining and estimation in the actual environment.

Thus, in the embodiment, the training device 10 can pre-train the CNN model 122 using a large amount of data not only including the input data and output data as the pre-training data with a fine granularity but also including input data and output data with a coarse granularity obtained in the actual environment.

Then, in the embodiment, the estimation apparatus 20 trains the CNN model 222 after the pre-training by weak retraining using a small amount of data obtained in the actual environment. Thus, the CNN model 222 can be generated that can perform estimation with high accuracy while avoiding overfitting, even when only a limited amount of data can be obtained in actual environment.

As described above, according to the embodiment, training data with which a CNN model can be appropriately pre-trained can be acquired, even when the size differs between the input data at the time of estimation in the actual environment and the input data for pre-training.

### Example 1

Next, a case where the present invention is applied to estimation of eyeball movement using EOG will be described as Example 1. The EOG is a method of estimating a line of sight direction, based on the fact that an eyeball is positively charged on the anterior side and negatively charged on the posterior side. For example, electrodes may be attached immediately above and immediately below an eyeball to measure the electrical potential. Then, when an increase in the electric potential immediately above and a decrease in the electrical potential immediately below the eyeball are measured, the anterior side of the eyeball, that is, the line of sight can be estimated to have moved upward.

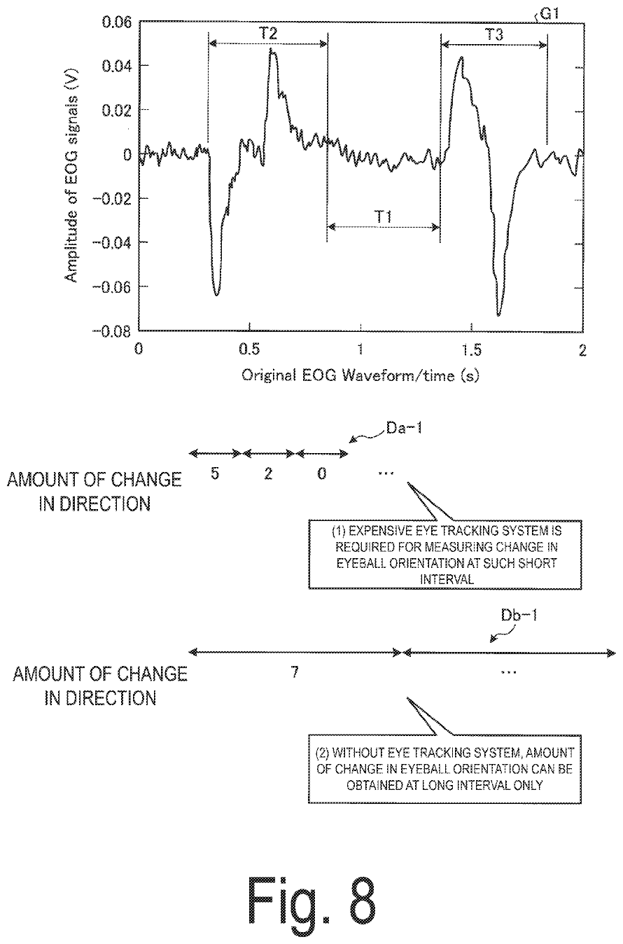

First of all, a related-art estimating eyeball movement method using EOG will be described. FIG. 8 is a diagram illustrating a related-art estimating eyeball movement method using EOG. A graph GI in FIG. 8 illustrates the time dependence of ocular potential measured by an AC EOG method. The graph GI is obtained by amplifying and recording an amount of change in the ocular potential. Here, in an interval T2, it can be estimated that the anterior side of the eyeball has shifted downward and stopped. This is because with the first change in the potential in the interval T2 being on the positive side, it can be determined that the negative potential of the posterior side of the eyeball has approached the electrode (shifted toward the upper side of the eyeball), and that the positive potential on the anterior side of the eyeball has moved away from the electrode (shifted toward the lower side of the eyeball). With the crest on the opposite side following this in the waveform, it can also be estimated that the eyeball has stopped immediately after the change in the direction. Furthermore, it can be estimated that the eyeball is not rotating in the interval T1 and that the anterior side of the eyeball is shifting upward in the interval T3.

The magnitude of a change in the direction of the eyeball can be estimated from the amplitude of a change in the potential. Specifically, the potential in the time zone (such as the interval T1) with no change in the direction of the eyeball is regarded as an offset value, and a higher crest corresponding to a change in the potential that has first occurred in an estimation interval thereafter indicates a larger change in the direction. In practice, to achieve sufficient accuracy, the magnitude is calculated by summing up (integrating) deviations of the potential in the region from the offset value, to calculate the magnitude of a change in the direction. In this process, when a waveform in a certain region as well as a change in an angle of the eyeball within the range can be acquired, the CNN model can learn the association therebetween, to be capable of estimating the orientation of the eyeball that has changed in the region on the basis of the waveform in a certain new region.

Here, in this estimation problem, the estimation target (the output) is a change in the orientation (sight position) of the eyeball. An eye tracking system that can acquire the absolute sight position is required for recognizing a change in the orientation of the eyeball. With an eye tracking system tracking the sight position in real time, the potential can be measured at a short time interval, whereby a change in the sight position within each interval can be acquired. For example, when the interval is 0.1 seconds, the pre-training can be performed with a change in the sight position within 0.1 seconds (see data Da-1) being the output.

In other words, measurement of a change in the eyeball orientation within such a short interval requires an expensive eye tracking system (see FIG. 8 (1)), which is not something that can always be prepared in the actual environment. Thus, in many cases, the eye tracking system is not used, and a change in the eyeball orientation is simply measured by making a user move his or her line of sight by a designated distance, and the training is performed using data (data Db-1 for example) in which the change is associated with the waveform of the potential within a designated time.

Unfortunately, without the eye tracking system, the amount of change in the eyeball orientation can only be obtained at a long interval (see (2) in FIG. 8). Specifically, it is possible to make the user implement calibration by “moving the line of sight by a designated distance within five seconds”, but the 0.1 second interval movement is impossible for the user. Thus, without the eye tracking, the real-time acquisition of the amount of change in the eyeball orientation cannot be achieved. All things considered, the amount of change in the eyeball orientation corresponding to a long time interval such as five seconds is used as the output.

In order to perform the estimation at a short time interval (such as 0.1 seconds), the output value measured at the short time interval needs to be retrained in the actual environment. However, even if the pre-training data is collected using the eye tracking system in a simulated environment, but the eye tracking system is difficult to provide in the actual environment. Thus, the retraining data corresponding to the granularity of the data for the pre-training is difficult to collect. Thus, in the related art, only data unsuitable for training for real-time estimation of the amount of change in the eyeball orientation has been available.

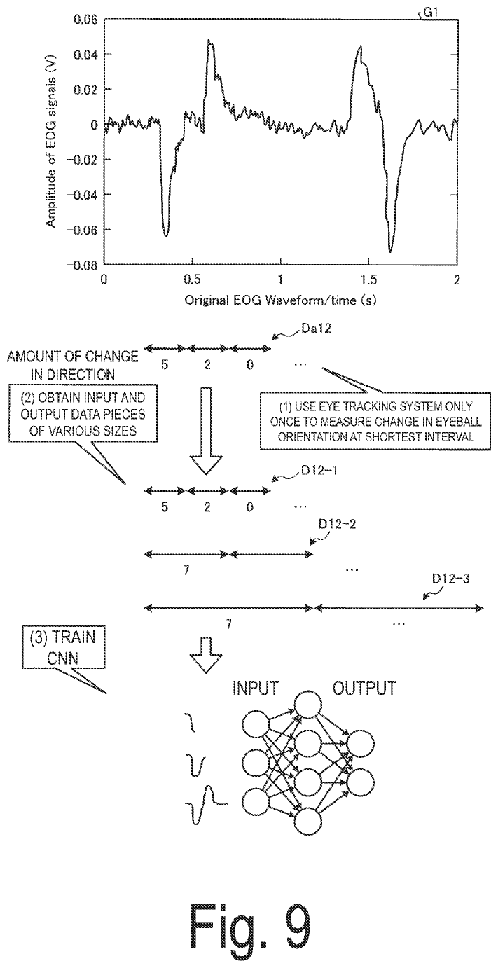

Next, an estimating eyeball movement method with an EOG according to this Example 1 will be described. FIG. 9 is a diagram illustrating pre-training for an eyeball movement estimation method using the EOG according to Example 1.

In Example 1, first of all, the pre-training data collection unit 131 of the training device 10 collects the pre-training data using an eye tracking system in a simulated environment. The pre-training data collection unit 131 collects the time-series data about the measurement values of the ocular potential of the user measured in an environment simulating an estimation environment for an eyeball movement as continuous input data, and collects the amount of change in the eyeball orientation as output data corresponding to the continuous input data.

For example, the pre-training data collection unit 131 measures the amounts of change in the eyeball orientation within the shortest time interval (see (1) in FIG. 9) by using the eye tracking system only once in the environment simulating a target environment for sight position estimation, thus collecting data. The collected data is data Da12 comprising input data pieces which are each the ocular potential waveform measured in each 0.1 interval for example, and output data indicating an amount of change in the eyeball orientation corresponding to the input data pieces. Note that if the sight position target is a monitor, the technique can be similarly applied to the monitor and if the target if a tablet, the technique can be applied to the tablet. In other words, the distance between the screen and the eyeball needs not to be adjusted to be a constant length, or physiological data of the same person needs not to be measured.

Then, the conversion unit 132 combines these input data pieces to generate sequential input data pieces of various sizes, and generates the output data pieces corresponding to the input data pieces of the sizes. The pre-training unit 133 makes the CNN model 122 learn the data (see (2) and (3) in FIG. 9).

Specifically, the conversion unit 132 combines the ocular potential waveforms (input data) each measured within 0.1 seconds, to generate waveforms of 0.2 second, 0.4 second, and 0.8 second intervals. Ten, the amounts of change in the eyeball orientation respectively corresponding to the ocular potential waveforms that are obtained by the conversion are obtained, whereby the pre-training data pieces (for example, D12-1 to D12-3) are obtained. For example, when the eye tracking system performs the measurement within each 0.1 second interval, the conversion unit 132 combines two consecutive ones of the ocular potential waveforms each captured at a 0.1 second interval, to generate input data as the ocular potential waveform at a 0.2 second interval, and obtains as output data, the amount of change in the eyeball orientation corresponding to the ocular potential waveform at the 0.2 second interval as a result of the combining.

Here, it is known that processing of extracting a feature of the input data is executed in a convolutional layer close to the input layer, and in a layer closer to the output layer, processing of estimating an output from the main feature extracted take place in the CNN. Of these, the processing of extracting the feature from the input (convolutional layer) can use the same model for different measurement environments as long as the measurement target is the same. When this processing is executed based on training, a large amount of input sequences with fine to coarse granularities are used, so that the convolutional layer with which feature extraction can be appropriately performed with an input sequence of a fine granularity input in the estimation environment can be generated.

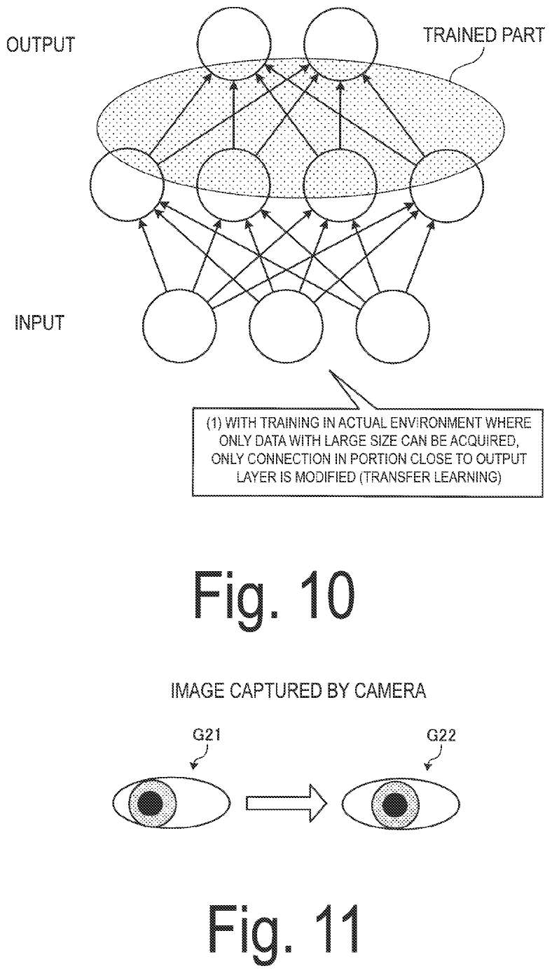

Next, the retraining in the actual environment to which the present embodiment is applied will be described. FIG. 10 is a diagram illustrating retraining for an eyeball movement estimation method using the EOG according to Example 1. In the actual environment, the eye tracking system is not used and a method of instructing an eyeball movement amount to a subject is performed. The CNN is retrained using the amount of change in the eyeball orientation acquired within a long time interval as an output, and a potential waveform as an input. In this process, when the retraining is performed in the actual environment where only data of a large size is available, the training (modification) is performed only one connection between several layers close to the fully connected output layer in the CNN (see (1) in FIG. 10).

With the pre-training, a convolutional layer with which features of the waveforms at various time intervals including short time intervals can be extracted is implemented. Here, only the fully connected layer for calculating the output from the main features extracted therewith is adjusted using data in the actual environment acquired at a long time interval. As described above, learning only using data acquired at a certain time interval results in a model specialized in such a time interval, to be capable of less sufficiently processing data acquired at other time intervals. In view of this, in Example 1, the training target is limited to the fully connected layer. Thus, a rough difference in the input/output relationship due to a difference between the simulated environment for the pre-training and the actual environment can be adjusted, while preventing the model as a whole from being only capable of processing data acquired in a long time interval.

### Example 2

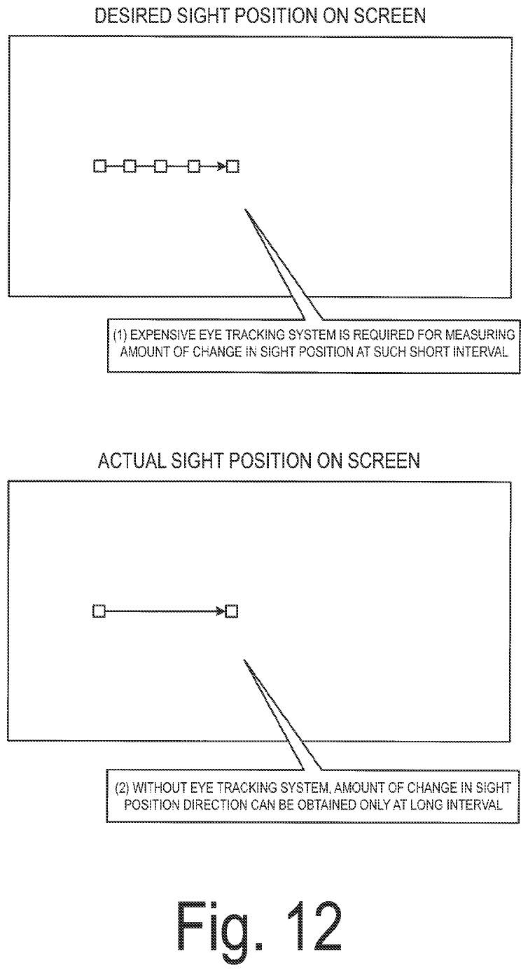

Next, as Example 2, a case in which the present embodiment is applied to sight position estimation using an image captured by a camera will be described. FIG. 11 is a diagram illustrating images captured by a camera. FIG. 12 is a diagram for explaining a sight position estimation method using an image captured by a camera in the related art.

In the sight position estimation using a camera, in many cases, images of the user's face are captured, and image processing is executed on such images G21 and G22 (see FIG. 11) to acquire the position of the pupil. The sight position estimation using a camera is implemented with the pupil position thus acquired associated with the sight position on the screen.

Now a case is considered where a change in eyeball orientation (sight position) is to be recognized from a camera image. An eye tracking system that can acquire the absolute sight position is required for recognizing a change in the direction of the sight position. With an eye tracking system tracking the sight position in real time, the images can be captured at a short time interval, and a change in the sight position within each time interval can be acquired. For example, when image capturing is performed at a 0.1 second interval, pre-training can be performed using the sight position at every 0.1 seconds as an output (see FIG. 12).

However, in order to acquire the sight position on the screen at a short time interval, an expensive eye tracking system is required which is not something that is available any time (see (1) in FIG. 12). Thus, in many cases, the amount of change in the direction of sight position is simply measured using a method of making the user move his or her line of sight by a designated distance, and the training is performed with the amount of change associated with the amount of movement of the pupil inside the image within a designated time. Thus, in a related-art without the eye tracking system, only the amount of change in the sight position has been obtainable at a long interval (see (2) in FIG. 12).

Thus, with this approach, the real-time acquisition of the amount of change in the eyeball orientation cannot be implemented. All things considered, the amount of change in the eyeball orientation corresponding to a long time interval such as five seconds is used as the output. Specifically, it is possible to make the user implement calibration by “moving the line of sight by a designated distance within five seconds”, but the 0.1 second interval movement is impossible for the user. In order to perform the estimation at a short time interval, such as a 0.1 second interval, the output values measured at the short time interval need to be learned. Thus, in the related art, there has been a problem in that only data unsuitable for learning for real-time estimation of the amount of change in the eyeball orientation is available.

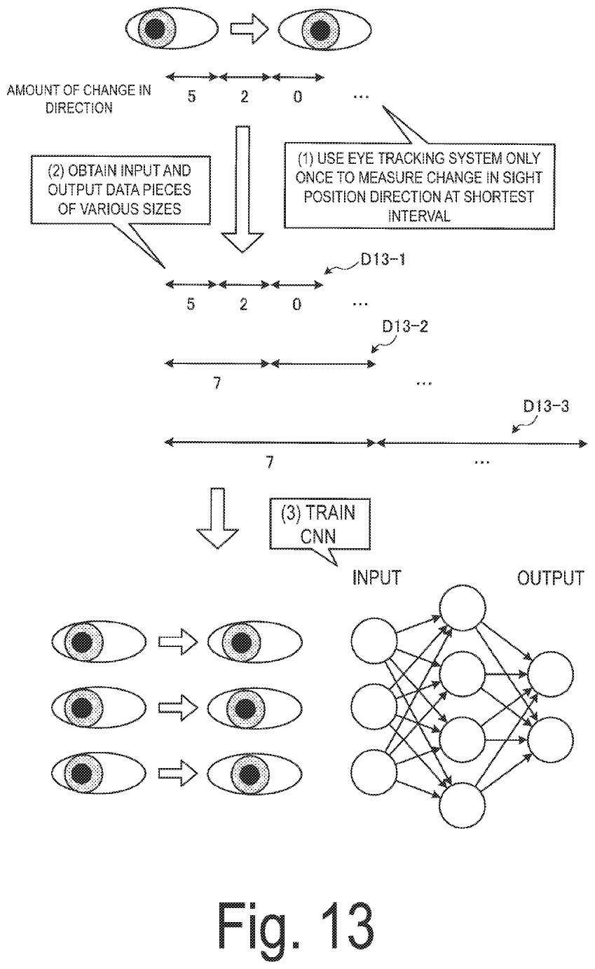

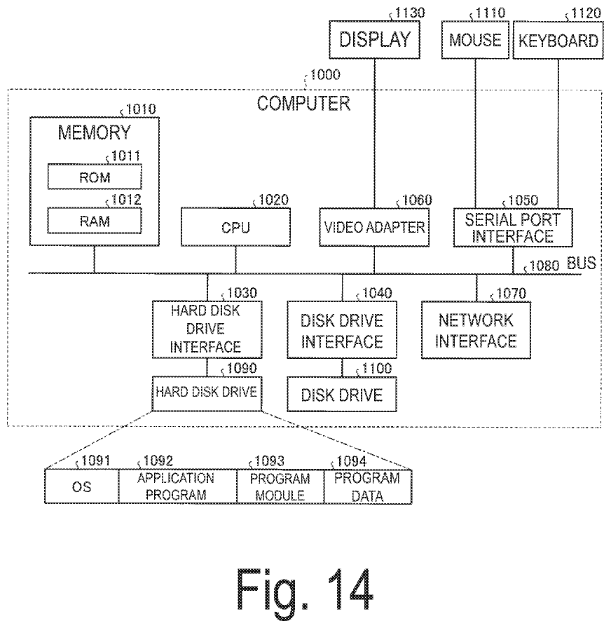

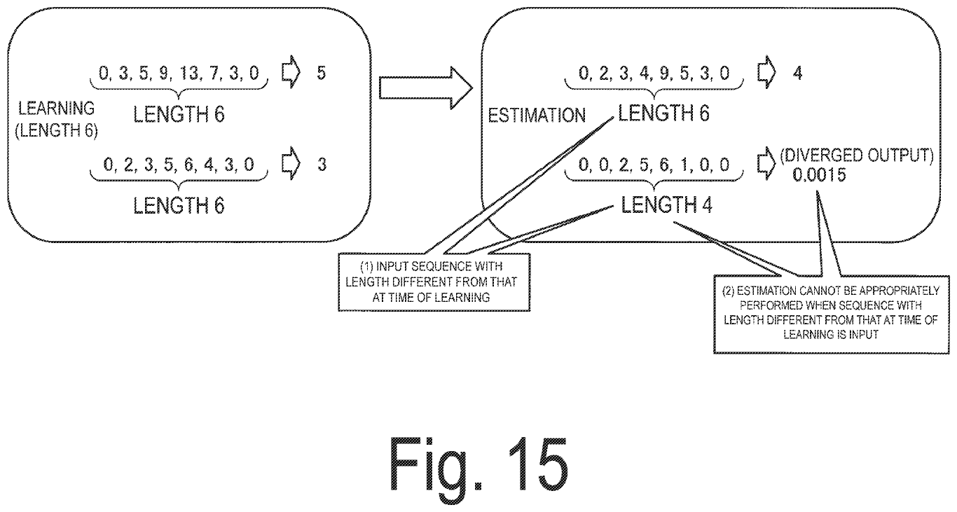

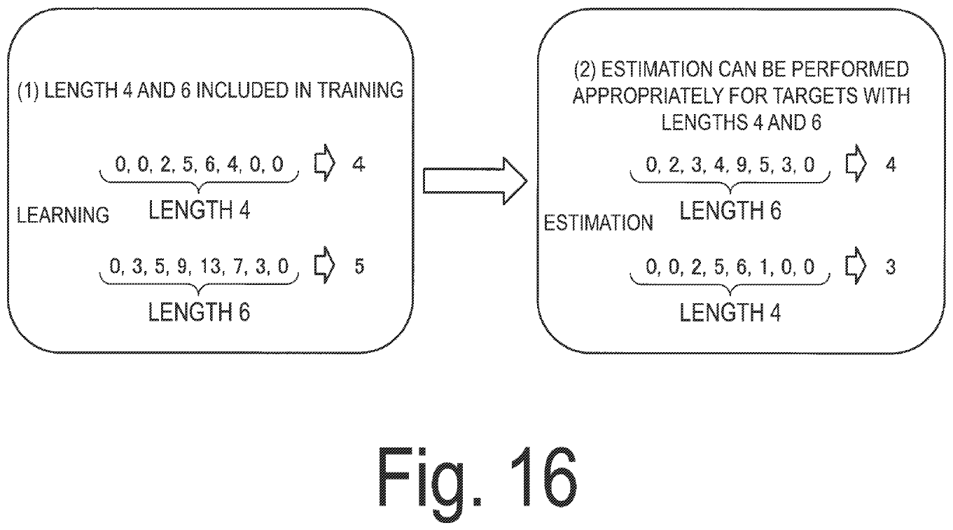

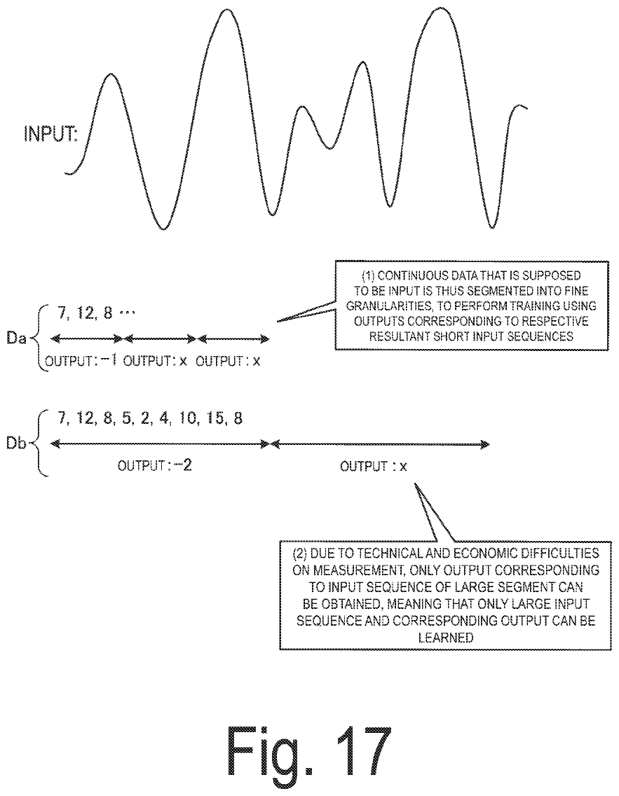

A sight position estimation method using an image captured by a camera according to Example 2 will be described. FIG. 13 is a diagram for explaining pre-training for the sight position estimation using an image captured by a camera according to Example 2.

In Example 2, first of all, the pre-training data collection unit 131 of the training device 10 collects, as continuous input data, the pupil position in the images of the user successively captured in an environment simulating an estimation environment for a sight position, and collects the amount of change in the direction of the sight position on the screen as the output data corresponding to the continuous input data.

Specifically, the pre-training data collection unit 131 acquires the images of the user captured at a short time interval using the eye tracking system as input data and measures the amount of change in the direction of the sight position corresponding to the input data, beforehand in the simulated environment simulating the target environment for sight position estimation (see (1) in FIG. 13). Note that if the sight position target is a monitor, the technique can be similarly applied to the monitor and if the target if a tablet, the technique can be applied to the tablet. In other words, the distance between the screen and the eyeball needs not to be adjusted to be a constant length, or physiological data of the same person needs not to be measured.

Then, the conversion unit 132 combines these input data pieces to generate sequential input data pieces of various sizes, and generates the output data pieces corresponding to the input data pieces of the sizes. The pre-training unit 133 trains the CNN model 122 with the data (see (2) and (3) in FIG. 13).

Specifically, the conversion unit 132 converts the image measured at a 0.1 second interval (input data) into an image at a 0.2 second interval, and obtains the amount of change in the sight position direction corresponding to each image obtained by conversion as the pre-training data (for example D13-1 to D13-3). For example, when the measurement is performed by the eye tracking system at a 0.1 second interval, the image at a 0.2 second interval is extracted as the input data from images captured at 0.1 second interval, and by obtaining, as the output data, the amount of change in the line of sight direction between the images extracted.

The CNN is not limited to the one dimensional input data described in relation to the EOG described in Example 1 (data in which one sensor value changes over time). Alternatively, two or higher dimensional data can be used as input. Thus, the pre-training unit 133 performs pre-training using an image that is the two-dimensional data (vertical×horizontal) changing over time, directly as the input for the CNN model 122.

Next, the retraining in the actual environment to which the present embodiment is applied will be described. In the actual environment, the eye tracking system is not used and a method of instructing an eyeball movement amount to a subject is performed. The retraining of the CNN is implemented using the amount of change in the direction of sight position acquired at a long time interval as an output, and a change in the image captured by a camera over time an input. In this case, as described above in Example 1, only the connection between several layers close the fully connected output layer in the CNN is trained (see FIG. 10).

### Example 3

Next, as Example 3, a case where the present embodiment is applied to an object movement amount estimation based on a measurement value obtained by an acceleration sensor will be described.

In this object movement amount estimation correction method, the CNN model perform pre-training using as input, time-series data acquired by an acceleration sensor while an object moves from one position to another, and using as output, the actual movement amount of the object. In such a case, to generate a CNN model capable of performing real-time estimation of the movement amount of an object, separate sensor information needs to be used to acquire the real-time object position, and the pre-training needs to be performed using the value thus obtained as output data and time-series data about the measurement values obtained by the acceleration sensor as input data. Examples of the separate sensor information include a position acquired using a tactile sensor. However, in the actual environment, a sensor separate from the acceleration sensor may available, and output values may not be obtained at a short time interval.

In such a case, the pre-training data collection unit 131 of the training device 10 collects the time-series data about the acceleration of the object measured in an environment simulating an estimation environment for an object movement as continuous input data, and collects the actual movement amount of the object as output data corresponding to the continuous input data. Specifically, in the simulated environment, the pre-training data collection unit 131 acquires, as pre-training data, a measurement value from an acceleration sensor obtained at a short time interval and a measurement value of an amount of object movement using another sensor that is different from the acceleration sensor.

Then, the conversion unit 132 combines these input data pieces to generate sequential input data pieces of various sizes, and generates the output data pieces corresponding to the input data pieces of the sizes. The pre-training unit 133 trains the CNN model 122 using the data. For example, in a case where the acceleration sensor and the amount of object movement are measured at a 0.1 second interval, the conversion unit 132 obtains the input data by converting two consecutive ones of the measurement values from the acceleration sensor measured at the 0.1 second interval into a value measured at a 0.2 second interval. Based on the object movement amount measured at a 0.1 second interval, the conversion unit 132 obtains, as the output data, the object movement amount corresponding to the measurement value obtained by the acceleration sensor obtained at the 0.2 second interval as a result of the conversion.

Then, in the actual environment, the estimation apparatus 20 performs the retraining using the object position information acquired at a longer time interval, to be capable of performing object movement amount estimation at a short time interval. In the actual environment, a method of recording a timing at which an object has moved over a certain position using a camera and the like may be employed.

The granularity of estimation of an output from an input by the CNN is often finer the better. However, when the estimation is desired to be performed with a fine granularity, some kind of solutions are required. Otherwise, the measurement of the input data and the output data with a fine granularity used for the pre-training would be the only option. On the other hand, a finer granularity of a certain measurement involves higher economical and technical difficulties. In such circumstances, the risk of the measurement facing economical and technical difficulties can be reduced, by avoiding the situations where fine granularity measurement is required.

In Examples 1 to 3, economical and technical difficulties in the measurement are avoided by reducing the need for measurement at fine granularity for output data used in retraining at the time of estimation. For example, as described in Examples 1 to 3, the present embodiment is applied to line of sight movement amount estimation using ocular potential, line of sight movement amount estimation using camera images, and estimation for other fields. As a result, the situation requiring real-time measurement often involving technical difficulties can be limited to the pre-training, and thus such a situation can be significantly avoided.

Also, in Examples 1 to 3, only the portion of the model close to the output layer is readjusted with a small amount of data acquired in the actual environment, so that the adjustment only requires a small amount of data. Under this condition, the amount of data may be insufficient depending on the data size. Specifically, only data of a certain size might be absent during the retraining. In view of this, as described in Examples 1 to 3, the training device 10 combines input data pieces measured with fine granularities at the time of pre-training so that sequences of various sizes are obtained, and the output data pieces corresponding to the input data pieces of various sizes are generated. Here, as described above in Examples 1 to 3, the training device 10 generates the pre-training data comprising input data of a certain size and output data as a learning target at the time of retraining. Thus, the pre-training and retraining can be appropriately executed even when only data of a certain size is absent at the time of retraining.

System Configuration or Like

The respective components of the devices that have been illustrated are functional and conceptual ones, and are not necessarily physically configured as illustrated. That is, a specific form of distribution and integration of the respective devices is not limited to that which is illustrated, and all or a portion thereof can be configured to be functionally or physically distributed and integrated in any units according to various load diagrams, use situations, and the like. Further, all or some of processing functions performed by each device may be realized by a CPU and a program that is analyzed and executed by the CPU, or may be realized as hardware based on a wired logic. The training device 10 and the estimation apparatus 20 according to the embodiments can also be realized by a computer and a program, and it is also possible to record the program in a recording medium and to provide the program through a network.

Further, all or some of the processes described as being automatically performed, among the processes described in the present embodiment, can also be manually performed, or all or some of the processes described as being manually performed can also be automatically performed by a known method. In addition, information including the processing procedures, control procedures, specific names, and various types of data or parameters illustrated in the aforementioned literatures or drawings can be arbitrarily changed unless otherwise specified.

Program

FIG. 14 is a diagram illustrating an example of a computer that realizes the training device 10 and the estimation apparatus 20 by executing a program. A computer 1000 includes, for example, a memory 1010 and a CPU 1020. Further, the computer 1000 includes a hard disk drive interface 1030, a disk drive interface 1040, a serial port interface 1050, a video adapter 1060, and a network interface 1070. These units are connected by a bus 1080.

The memory 1010 includes a read only memory (ROM) 1011 and a RAM 1012. The ROM 1011 stores, for example, a boot program, such as a basic input output system (BIOS). The hard disk drive interface 1030 is connected to a hard disk drive 1090. The disk drive interface 1040 is connected to a disk drive 1100. A detachable storage medium such as a magnetic disk or optical disk, for example, is inserted into the disk drive 1100. The serial port interface 1050 is connected to, for example, a mouse 1110 and a keyboard 1120. The video adapter 1060 is connected to a display 1130, for example.

The hard disk drive 1090 stores, for example, an OS 1091, an application program 1092, a program module 1093, and program data 1094. In other words, a program defining each process of the training device 10 and the estimation apparatus 20 is implemented as the program module 1093 in which a code executable by the computer 1000 is described. The program module 1093 is stored in, for example, the hard disk drive 1090. For example, the program module 1093 for executing the same process as that of a functional configuration in the training device 10 and the estimation apparatus 20 is stored in the hard disk drive 1090. The hard disk drive 1090 may be replaced with a solid state drive (SSD).

Further, setting data used in the process of the embodiment described above is stored as the program data 1094 in the memory 1010 or the hard disk drive 1090, for example. The CPU 1020 reads the program module 1093 or the program data 1094 stored in the memory 1010 or the hard disk drive 1090 into the RAM 1012 as necessary, and executes the program module 1093 or the program data 1094.

The program module 1093 or the program data 1094 is not limited to being stored in the hard disk drive 1090, and may be stored, for example, in a detachable storage medium and read by the CPU 1020 via the disk drive 1100 or the like. Alternatively, the program module 1093 and the program data 1094 may be stored in another computer connected via a network (such as a LAN or a Wide Area Network (WAN)). The program module 1093 and the program data 1094 may be read from another computer via the network interface 1070 by the CPU 1020.

Although embodiments to which the invention made by the present inventor has been applied have been described above, the present invention is not limited to the description and the drawings that form part of the disclosure of the present invention according to the embodiments. That is, all other embodiments, examples, operation techniques, and the like made by those skilled in the art on the basis of the present embodiments are included in the scope of the present invention.

### REFERENCE SIGNS LIST

1 Estimation system
10 Training device
11, 21 Communication processing unit
12, 22 Storage unit
13, 23 Control unit
121 Pre-training data
122, 222 CNN model
130 Preprocessing unit
131 Pre-training data collection unit
132 Conversion unit
133 Pre-training unit
20 Estimation apparatus
221 Retraining data
231 Retraining data collection unit
232 Retraining unit
233 Estimation unit

1. A preprocessing apparatus comprising:
a collection unit, including at least one processor, configured to collect pre-training data comprising continuous input data measured in an environment simulating an estimation environment and output data corresponding to the continuous input data; and
a conversion unit, including at least one processor, configured to convert the continuous input data into continuous input data pieces of a plurality of sizes including a size larger than the input data, convert the output data corresponding to the continuous input data into output data pieces respectively corresponding to the continuous input data pieces of the plurality of sizes, and output the continuous input data pieces of the plurality of sizes and the output data pieces as training data.

2. The preprocessing apparatus according to claim 1, wherein the conversion unit converts the continuous input data in accordance with a distribution in which an amount of at least input data for retraining in the estimation environment is equal to or larger than an amount of input data of a size different from a size of the input data for the retraining.

3. The preprocessing apparatus according to claim 2, wherein
the distribution corresponds to a probability distribution in which the amount of the input data for the retraining is larger than the amount of the input data of the different size, and
the probability distribution is a convex probability distribution with a size of input data used in the estimation environment at center of the distribution.

4. The preprocessing apparatus according to claim 1, wherein the collection unit collects the pre-training data including a size of at least one input data for retraining in the estimation environment as a size of the continuous input data, and comprising a dataset including an indicator enabling a pre-training algorithm to determine data of a size that is equal to the size of the input data for retraining in the estimation environment.

5. The preprocessing apparatus according to claim 1, wherein the collection unit collects time-series data about measurement values of an ocular potential of a user measured in an environment simulating an estimation environment for an eyeball movement as the continuous input data, and collects an amount of change in an eyeball orientation as output data corresponding to the continuous input data.

6. The preprocessing apparatus according to claim 1, wherein the collection unit collects, as the continuous input data, a pupil position of a user in images successively captured in an environment simulating an estimation environment for a sight position, and collects, as the output data corresponding to the continuous input data, an amount of change in a direction of the sight position on a screen.

7. The preprocessing apparatus according to claim 1, wherein the collection unit collects time-series data about measurement values of an acceleration of an object measured in an environment simulating an estimation environment for a movement of the object as the continuous input data, and collects an amount of change in an actual movement amount of the object as output data corresponding to the continuous input data.

8. A preprocessing method performed by a preprocessing apparatus, the method comprising:
collecting pre-training data comprising continuous input data measured in an environment simulating an estimation environment and output data corresponding to the continuous input data; and
converting the continuous input data into continuous input data pieces of a plurality of sizes including a size larger than the input data, converting the output data corresponding to the continuous input data into output data pieces respectively corresponding to the continuous input data pieces of the plurality of sizes, and outputting the continuous input data pieces of the plurality of sizes and the output data pieces as training data.

9. A non-transitory computer readable medium comprising a preprocessing program that causes a computer to perform operations including:
collecting pre-training data comprising continuous input data measured in an environment simulating an estimation environment and output data corresponding to the continuous input data; and
converting the continuous input data into continuous input data pieces of a plurality of sizes including a size larger than the input data, converting the output data corresponding to the continuous input data into output data pieces respectively corresponding to the continuous input data pieces of the plurality of sizes, and outputting the continuous input data pieces of the plurality of sizes and the output data pieces as training data.
